(19) United States
(12) Patent Application Publication
(10) Pub. No.: US 2004/0073104 A1
(43) Pub. Date: Apr. 15, 2004

(54) Enhanced pickup-electrode

(76) Inventors: Riccardo Brun del Re, Ottawa (CA); Izmail Batkin, Gloucester (CA)

Correspondence Address: David J French, Station D, PO Box 2486, Ottawa, K1P 5W6, CA

(21) Appl. No.: 10/468,776

(22) PCT Filed: Feb. 22, 2002
(86) PCT No.: PCT/CA02/00209

(30) Foreign Application Priority Data
Feb. 23, 2001 (WO) PCT/CA01/00217

(51) Int. Cl. A61B 5/0408

(52) U.S. Cl. 600/372

(57) ABSTRACT

A bio-electrode (54) for obtaining ECG's and EEG's is enhanced by a cushioning layer (51) placed intermediate the body (3) and electrode (54). This enhancement layer (51) can carry and release water or moisture into the electrode-to-body interface. Moisture provided to this interface is ionically conductive, reducing tribo-electric noise. A bio-electrode so enhanced can obtain signals through clothing.

### FIELD OF THE INVENTION

This invention relates to electrodes for bio-electric field sensing. In particular, it relates to electrodes for electro-cardiograms (ECG), electro encephalograms (BEG), heart rate monitoring systems and the like.

### BACKGROUND TO THE INVENTION

Electrodes for the pickup of bio-electric signals can be categorized as being either ohmic or capacitive. Ohmic electrodes are resistively coupled to the body. Capacitive electrodes are capacitively coupled. Almost all bia-electrodes presently in use are ohmic. Ohmic electrodes may further be classed as ‘gel’ electrodes if they utilize an electrolytic gel, jelly, or paste which is placed in contact with the skin during use to maximize electrical coupling to the body.

Many types of gel electrodes are disposable and possess self-contained gels in the form of ‘solid’ gel or ‘wet’ gel structures. These have limited shelf life, are not re-usable because the electrolytic gels tend to be fragile, sticky, and susceptible to drying out after exposure to air. Further, these types of electrodes utilize strong skin adhesives to ensure that intimate coupling to the body is established. Other types of gel electrodes, such as suction cup electrodes, require the user to smear electrolytic gel or paste on the electrode prior to each application and are inconvenient for this reason. Suction cup electrodes are also incompatible with free body movement.

‘Gel-free’ electrodes, sometimes called ‘dry’ electrodes, have been proposed for re-usable electrode applications. Such electrodes can be used with electrolytic gels or pastes but can also be used without gels or pastes via simple mechanical contact with the skin. Gel-free use is attractive because it avoids the need for adhesives, fragile gels, or manual gel application.

Dry electrodes of the prior art occur in two functional categories: ohmic and capacitive. Dry ohmic electrodes obtain body signals by direct contact between a conductive element and the skin. Capacitive electrodes obtain body signals via electrostatic induction or ‘displacement currents’ which are mediated through an insulating layer positioned between the body and a conductive element that serves as a capacitor plate. Typically, the insulating layer is intimately bonded to the conductive element in a way that precludes ohmic contact between the conductive element and the body.

While the present invention is, in one variant, particularly suited for use with capacitive electrodes, it is also applicable for use with dry ohmic electrodes. The invention relates to an improvement over the prior art in the form of a body-engaging electrode interface which improves the coupling between bio-electrodes and the body.

Capacitive electrodes of the prior art were generally, designed for high coupling operation via direct contact between the electrode dielectric and the skin. Prior art capacitive electrodes generally possess insulating (dielectric) layers with smooth, hard surfaces. In the past, the selection of materials for the insulating layer of capacitive electrodes was restricted by the perceived requirement of achieving a high value of capacitive coupling to the body. The nominal capacitive coupling between the electrode and the body is given by the approximate relationship C=eA/d where C is capacitance, e is the dielectric constant of the insulator, A is the area of the electrode, and d is the thickness of the insulator. This formula shows that a high value of capacitive coupling may be obtained using a thin insulating layer and an intimate degree of coupling to the body.

In order to accomplish a high capacitive coupling, prior art capacitive electrodes were generally restricted to the use of specialized insulating materials which could be deposited, sputtered, grown, or otherwise intimately bonded to the electrode conductive element as a thin, homogenous, contiguous, impermeable and stable dielectric layer. Dielectric materials used in prior art technologies have been typically hard, brittle and non-moisture absorbent such as glass, aluminum oxide or tantalum oxide. Other insulating materials used in the prior art include films such as Mylar films which are moderately flexible but these suffer the drawback of mechanical instability in the electrode-to-body coupling resulting from the surface hardness and smoothness of the insulating layer. Such prior art electrodes are not self-stabilizing in their mechanical contact with the body and tend to slide on dry skin or hairy skin. This causes noise in the output signal. Although prior art electrodes were not premised for use on clothing, the above problems are even further magnified when attempts are made to utilize such capacitive electrodes over clothing fabrics.

High coupling capacitive electrodes of the prior art possess a further, and fundamental drawback related to the thinness of their dielectric layers. The main reason for signal degradation with prior art capacitive electrodes is that the intervening layers are electrically equivalent to the presence of a ‘parasitic’ capacitance placed in series between the electrode dielectric and the signal source. For prior art capacitive electrodes with thin dielectric layers this parasitic capacitance can easily dominate the electrode-to-body coupling.

For example, clean, dry, matted chest hair beneath an electrode can easily create a gap of 0.1 mm with effective dielectric constant of approximately one. This is equivalent to a parasitic capacitance of approximately 9 pF/cm². The dry horney layer of the skin (stratum corneum) can typically create parasitic capacitances in the range of 50 pF/cm² to 100 pF/cm² depending on the age of the skin, its moisture content and the skin type. All the above parasitic capacitance values are smaller than the nominal capacitance values of typical prior art high coupling capacitive electrodes i.e. which range between 100 pF/cm² to 10⁴ pF/cm² with many examples even higher.

Because series capacitor networks are dominated by the smallest value capacitor, the net electrode-to-body coupling for typical prior-art electrodes with thin dielectrics applied on dry or hairy skin often approximates to the parasitic capacitance value and not the electrode nominal capacitive value. This has been problematic because the sensor electronics generally associated with prior art capacitive electrodes are premised in its design on there being a fixed, predictable and substantial capacitive coupling to the body. Any departure from this expected coupling value results in loss of signal amplitude and loss of low-frequency response. Even with intimate contact to hairless skin, unpredictable changes in capacitive coupling of prior art electrodes can result from the build-up of sweat or moisture in the skin horney layer. For similar reasons, the net coupling can be pressure-dependent and motion-sensitive. Such effects have greatly limited the use of prior art capacitance electrodes with thin dielectrics. Similar disadvantages arise in prior art capacitive electrodes with extremely high dielectric constants and their correspondingly designed electronics.

As a separate phenomenon, both capacitive and ohmic dry electrodes suffer from noise when applied on dry skin or hairy skin. This is believed to be due to changes in resistance and to resistive (current) noise in the contact between the electrode and the body, and to noise generated by static charges arising from the mechanical contact between the electrode and the hair or dry skin with which it is placed in contact. Triboelectric noise also arises when attempts are made to obtain signals using capacitive electrodes over clothing fabric. This noise voltage can easily be orders of magnitude larger than the desired body signal and noise from this source can be highly motion sensitive.

Triboelectric noise is usually the most significant noise factor in high-impedance sensors used in conjunction with capacitive electrodes. However, and in contrast, ohmic electrodes suffer from noise induced by electrochemical phenomena called ‘Nernst potential’ or ‘half-cell’ or ‘battery potential’ effects. This refers to the molecular, charged bi-layers which are spontaneously created at any interface between a metallic conductor and an electrolyte. These effects are a source of both spontaneous and motion-induced noise arising as a result of chemical reactions and mechanical disturbances at the electrode-to-skin interface. Body fluids such as sweat are electrolytes i.e. their conductivity arises from ionic charge mobility.

Traditional ohmic ‘gel’ electrodes typically possess a conductive element with a silver chloride coating which is embedded in a viscous electrolytic gel. The gel serves as an electrolytic layer which prevents direct contact between skin and any metallic elements thus avoiding the formation of an unstable skin-to-metal ½-cell in favour of a stable ½-cell at the chloridated metal-to-gel interface. The latter interface is electrically and mechanically stabilized by virtue of its stable local chemistry and its physical confinement within the chloridated metal coating and surrounding gel. Intimate contact of the gel with the body is maintained by a surrounding strip of adhesive which further minimizes the disturbance of the body-to-gel and gel-to-metal interfaces. The use of such adhesively stabilized gel electrodes is uncomfortable for patients subject to long-term ECG monitoring and is impractical in pre-formatted arrays of electrodes, and for electrodes which must operate re-usably by simple mechanical contact between electrode and the body. Furthermore, conventional gel electrodes are not practical for use over clothing.

A need exists for a bio-electrode possessing a body-engaging layer which is both comfortable and which provides stable pickup of body electric signals.

The invention in its general form will first be described, and then its implementation in terms of specific embodiments will be detailed with reference to the drawings following hereafter. These embodiments are intended to demonstrate the principle of the invention, and the manner of its implementation. The invention in its broadest and more specific forms will then be further described and defined in each of the individual claims which conclude this Specification.

### SUMMARY OF THE INVENTION

According to one aspect of the invention a bio-electrode of either ohmic or capacitive type for measuring localized signals arising on the surface of a body is fitted with a coupling layer on the body-facing surface. In the case of a capacitive electrode, this layer is placed against the dielectric surface of the electrode. In the case of an ohmic electrode, the layer is placed in contact with the conductive surface, which may optionally be chloridated, which conveys signals to the sensing circuitry. This coupling layer is self-stabilizing i.e. non-slippery on the body without the need for adhesives. Preferably, it should be sufficiently soft to conform to small-scale body features such as skin irregularities and hair and to provide a cushion against motion. Thus the coupling layer possesses a softness and texture which establish a comfortable engagement to the body while acting as a cushion or shock absorber minimizing mechanical motions at the interfaces between the body and the layer, and between the layer and the electrode components remote from the body inter-face.

The coupling layer preferably has moisture carrying capacity and the ability to release water or moisture into the electrode to body interface. It is also desireable that the coupling layer have the capability of “buffering” changes in the moisture state existing at the interface. When moistened with ionically conductive liquids such as ordinary tap water possessing ordinary ionic impurities, or saline solution or the like, the layer is able to support a stable pathway for spontaneous ionic conduction between the body and the body-facing surface of the electrode.

The layer should also preferably be, according to the invention, moisture absorbing when dry as well as moisture emanating when wet. This enables the layer to moderate changes in the moisture arising between the electrode and the body as would be induced by sweating. This moisture ‘buffering’ property helps to minimize sweat-induced changes in interface capacitance and resistance, and sweat-induced ½-cell potential effects in the case of metallic components. Moisture emanated from the moistened layer also serves to shunt parasitic interface capacitances resulting from dry skin, hair, or clothing layers and to quench triboelectric effects.

For brevity in what follows the coupling layer will be called the ‘enhancer’ or ‘enhancement layer’ and an electrode possessing an enhancer shall be called an enhanced capacitive electrode or an “enhanced ohmic” electrode.

The mechanical properties of the enhancer allow it to stabilize the mechanical coupling between the electrode and the body by engaging with small-scale body curvatures, hair, and surface skin features. The moisture-carrying capacity of the enhancer provides it with the ability to absorb, store, and evolve moisture thereby ensuring delivery preferably in an even distribution, of moisture between the electrode and body. When moistened the layer can emanate moisture into the electrode-to-body interface, thus quieting tribo-electric noise and improving the electrical coupling between the electrode and the body signal source when applied on dry skin, hairy skin, or over clothing.

The desirable electrical features of the enhancer are that it be substantially conductive when moistened i.e. that it displays a bulk resistance less than 1 Mohm and preferably less than several kilo-ohms This conductivity may be permanent, as where a conductive material is employed, or may be induced by the addition of ordinary water containing conduction-supporting minerals, electrolytes such as saline solutions, or by the presence of ambient moisture or sweat. The enhancer's mechanical conformability and electrical conductivity serve to create an ohmic link between the body signal source and the electrode. In the case of capacitive electrodes the enhancer serves to shunt parasitic interface capacitances arising from the horney layer of the skin and from any hair, clothing fabric and air gaps. This helps to stabilize the net capacitive coupling between the electrode and the body over time and ensures that this coupling is nearer to the nominal capacitance of the electrode independently of parasitic capacitances arising from dry skin, hair, and clothing.

By way of an analog, whereas prior art capacitive electrodes are, in essence, simple capacitors for bio-potential pickup, enhanced capacitive electrodes of the present invention might be seen to be in the nature of padded electrolytic capacitors for bio-potential pickup wherein the padded (enhancement) layer possesses conductivity sufficient to present the desired body signal evenly to the electrode dielectric layer while at the same time interfacing comfortably with the human body.

As the enhancer's conductivity is preferably derived from ionic conductivity eg. via ions or charged-carrying impurities, noise-inducing electrochemical potentials at the enhancer-to-body interface are avoided or reduced. The conductivity of the enhancer can arise temporarily merely as a result of added moisture particularly when salts or ions are already present in the enhancer. A preferred embodiment especially suited to ohmic electrodes employs an enhancer containing a highly absorbent material such as a superabsorbent, water-releasing polymer to slowly release an effective quantity of ionically conductive moisture into the body-to-electrode interface.

In a preferred case applicable to capacitive electrodes the enhancer layer is intimately bonded to, or incorporated into the structure of the electrode. One example in the case of a capacitive electrode is an enhancer that is permanently glued or fused onto an already present insulating layer. Another example is a pre-fused enhancer-insulator structure manufactured as a single unit such as a sponge or a flocced fabric with an impermeable insulating backing such that the insulating backing can be used as the actual electrode insulating layer and the sponge or fabric is used as the enhancer.

As another embodiment of the invention, an enhanced capacitive or ohmic electrode or pickup system can be constructed as two autonomous, independent units—i.e. as a plain electrode or electrode array and as a separate enhancer or enhancer array which is manually positioned between the non-enhanced electrode array and the patient's body by the user. Such an enhancer may be held in position against the electrode by a positioning means such as a fabric pocket, or by an adhesive applied between the enhancer and the electrode. This allows for low-cost, re-usable or disposable ‘peel-and-stick’ type of enhancers which are hygenic.

Examples of useful enhancer materials include conventional fabrics such as cotton denim, rayon, and satin-finished polyester as well as artificial chamois and other equivalent materials. Such an enhancer layer can be intimately bonded to a pre-existing electrode by way of either a conductive or insulating adhesive. The resulting enhanced electrode surface possesses a surface texture which provides grip and which accommodates small scale body irregularities e.g. skin imperfections and hair. Advantageously, but not necessarily, the fabric or other microporous enhancer can be pre-treated to provide. an electrolyte as by soaking it with an electrolytic solution and then drying it to leave a salty residue that will render it conductive when wetted. Useful electrolyte solutions include but are not restricted to a mild solution of sodium, potassium, calcium lithium and magnesium chloride, or mixtures thereof with appropriate ionic conductivity and deliquescent properties.

A preferred variant of the invention which is useful for the pickup of bio-electric signals of both the ohmic and capacitive types is based on an enhancer that contains a highly water-absorbing material or matrix, eg. ‘superabsorbent’ materials. Superabsorbents are defined as, dry materials which can spontaneously absorb substantial quantities of an aqueous fluid at a rate of at least twenty times their own weight—(Absorbent Polymer Technology, Studies in Polymer Sciences 8, Elsevier, 1990). A preferred class of superabsorbents are the superabsorbent polymers that form gels in contact with water. These non-soluble acrylic polymers polyacrylate/polyacrylamide-based compounds commonly used in hygiene products including diapers. A preferred superabsorbent is based on cross-linked sodium or potassium polyacrylate/polyacrylamide (CLP). These substances are made available in granular form as for example by the Terawet company. An enhancer based on CLP granules contained in a cloth bag or pouch can be wetted with ordinary tap water-to render it cushion-like, moisture emanating, and possessing sufficient conductivity to manifest all the electro-mechanical features required to realize the advantages of the invention i.e. softness, signal stabilizing effects, and noise reduction. Once saturated, the CLP has the advantage of releasing moisture in a slow, controlled manner and over long periods of time. Although CLP is a preferred variant, the moisture containing matrix may be comprised of any type of sponge or porous material or carrier that will absorb water when dry and slowly release water when wet.

When used with a capacitive electrode applied over clothing, moisture from the enhancer can penetrate clothing layers and provide a conductive layer which serves as a conductive bridge between the body signal source and the electrode elements—i.e. thereby shunting the parasitic interface impedance arising from the clothing and from dry skin or hair beneath it. Moisture also stabilizes the coupling of a electrode to the body by reducing triboelectric noise at the electrode-to-body interface, providing a discharge pathway to the body for local accumulations of charge.

The application of the invention in its capacitive variant is facilitated by employing a capacitive electrode sensing system with a very low input capacitance. This enables the use of a low capacitance coupling to the body with advantages as next described.

It has been found, as described in PCT application PCT/CA00/00981, the contents of which are adopted herein by reference, that a capacitive pickup electrode system may be advantageously operated in the low-capacitance region of the relationship C=eA/d where the capacitance C is relatively insensitive to variations in separation gap d,—e.g. a 0.1 mm increase in the displacement of the electrode causes less than a 50 percent decrease in C or equivalent rate. An electrode system may be operated in this range by providing an electrode and sensing circuit designed to operate in the low capacitive coupling regime; such an electrode may have a dielectric layer with a low dielectric constant and a relatively increased thickness—e.g. 0.1 mm to 1.0 mm or more. Alternately, a series capacitance of low value—e.g.—5 to 40 picoFarads can be placed in series with the pickup electrode in its connection to a high impedance input amplifier circuit.

This arrangement enables use of arbitrary dielectric materials as opposed to highly specialized dielectrics as in the prior art. This decreases the signal sensitivity to variations in individual electrode characteristics and allows greater scope in the selection of electrode dielectric materials and manufacturing processes. In contrast to the prior art which was restricted to using specialized dielectrics, a much greater variety of dielectrics can now be used due to low-coupling demands placed on the pick-up electrode. Capacitive electrodes can be made flexible by using highly flexible dielectrics. These can provide a more stable contact and comfortable fit on the body. Unlike the prior art, capacitive electrodes employed under the invention can be made more robust and scratch-resistant by using stronger or thicker dielectrics. Operation in a low-coupling regime enables enhancers to be bonded or adhered to the capacitive electrode dielectric by use insulating bonding agents and adhesives without causing a degradation in the electrode behaviour.

A further benefit of using a capacitive electrode sensing system with very low input capacitance is that signal pickup is possible with a relatively low level of intimacy of contact to body. As described in the introduction, this is due to the fact that low-coupling electrodes with relatively thick dielectrics can possess nominal capacitance values that are lower than the typical parasitic capacitances on the body thus remaining relatively insensitive to the presence of dry skin, hairy skin, or clothing. Such electrodes can also remain insensitive to temporal effects of sweat permeation into the horney layer. Operation in the low-coupling regime also allows for the acquisition of signals through clothing fabric layers and facilitates the use of enhancement layers of the present invention.

Appropriate realizations of the invention can be used to construct heart-rate and ECG electrodes capable of obtaining signals in locations not practical with non-enhanced capacitive or conventional ohmic electrodes. This includes over-clothing pickup, waist level pickup, pickup from the ear, as for example, if electrodes of the invention are built into ear-phone cushions, and pickup from other body parts such as the back or the shoulders when electrodes of the invention are placed under or built into straps, backpacks, belts, or clothing-like supports.

In this manner, a bio-electrode system may be provided that is convenient and comfortable for the user, and less susceptible to disruption from noise.

The foregoing summarizes the principal features of the invention and some of its optional aspects. The invention may be further understood by the description of the preferred embodiments, in conjunction with the drawings, which now follow.

### BRIEF DESCRIPTION OF THE DRAWINGS

FIG. 1A is a combined pictorial/electrical schematic depiction of a single pick-up of the invention in position adjacent to a body whose electrical field is to be sensed. The voltage divider network is capacitively coupled to the body at both ends and drives an operational amplifier.

FIG. 1B is a conventional electrical schematic corresponding to the input portion driving the amplifier of FIG. 1A.

FIGS. 1C and 1D are the schematics of FIGS. 1A and 1B with the added presence of a series capacitor in the amplifier input.

FIG. 2A is FIG. 1A with the substitution of a resistive, conductive coupling to the body at one end of the voltage divider network.

FIG. 2B is a conventional electrical schematic corresponding to FIG. 2A.

FIG. 3 is an electrical schematic for a dual pick-up electrode configuration, based on the pick-up of FIG. 1A, with signals being fed to a differential amplifier, but with dual, parallel Schotkey diodes as input leakage resistors.

FIG. 4 is an expanded electrical schematic of the circuit of FIG. 3 with the additional presence of an amplifier and optical coupler to provide electrical isolation.

FIG. 5 is a graph showing the change of capacitance of pick-up electrodes with various surface areas as a function of separation distance for the electrodes.

FIG. 6 is a graph showing the percentage change in capacitance for a 0.1 mm change in electrode-to-body gap distance as a function of nominal electrode-to-body gap distance over a range of 0.0 to 1.0 mm, assuming the body acts as a perfect electrode.

FIG. 7 is a plan view of an electrical circuit corresponding to FIG. 4 laid-out in a belt to be worn over the chest of a patient.

FIG. 8 is a pictorial depiction of the belt of FIG. 7 in place over the chest of a patient.

FIG. 9 is a pictorial version of a garment worn by a patient that carries four pick-up electrodes.

FIG. 10 is a graph of total effective coupling capacitance between the sensed body and the input to the amplifier of the sensor, plotted as a function of the separation distance of the electrode from the surface being sensed. Three curves are shown, two with a limiting series capacitor present and one with no limiting capacitor present.

FIG. 11 is similar to FIG. 10 but with the vertical scale for the input capacitance increased by a factor of ten and showing one curve with and one curve without a limiting capacitor present.

FIG. 12 is a cross-sectional side view of a capacitive electrode with on-board electronics equipped with an enhancer or enhancing layer according to the invention positioned at the body interface for the electrode.

FIG. 12a is a side of an ohmic electrode carrying a cross-section enhancer.

FIG. 12b is a schematic of the electrical circuit used to model FIG. 12a.

FIG. 12c is a plan view of an existing heart rate ohmic pick-up and matching enhancer dimensioned to fit thereon.

FIG. 12d is a cross-sectional side view of an alternate ohmic electrode with enhancer to that of FIG. 12a.

FIG. 13 is a graph of the approximate signal to noise ratios for enhanced capacitive electrodes, both pre-moistened and ostensibly dry, as a function of increasing numbers of layers of a cotton T-shirt fabric over the skin of a human body.

FIG. 14 is a graph of R-peak amplitude seen in the same data set used for FIG. 13 i.e. for enhanced capacitive electrodes, both pre-moistened and ostensibly dry, as a function of increasing numbers of folded layers of cotton T-shirt material over human skin.

FIG. 15 is a dual, real-time display comparing differential ECG-like signals obtained using two pairs of electrodes placed side-by-side on the unprepared skin of a test subject and held in position using an elastic strap. The pickup geometry was an approximate MCL₃ configuration employing right sub-clavian and V₃ electrode sites. The bottom trace shows the conventional wet-gel electrode signal while the top trace shows an un-enhanced low-coupling capacitive electrode signal. Nominally identical differential amplifiers were used for each electrode pair.

FIG. 16 is a graph showing a differential pickup signal obtained by placing enhanced capacitive electrodes with moistened enhancers held in place by a chest belt over unprepared skin of a test subject. The electrodes were held in position several centimeters below each nipple of the test subject by use of an elasticized fabric strap placed around the chest. Electrodes were connected to the lead-one inputs of a commercial ECG machine. All other ECG inputs were grounded to the subject body-reference via a stainless steel plate at the right foot.

FIG. 17 is a graph of the chest-belt configuration of FIG. 16 but showing enhanced capacitive signal pickup over an initially dry, woven cotton golf-shirt covering the skin of a human body.

FIG. 18 is a graph as in FIG. 17 showing pickup using an ostensibly dry enhancer over a dry cotton golf-shirt covering the skin of a human body.

FIG. 19 is a graph showing a signal obtained over a dry, cotton golf-shirt covering the skin of a human body using an unenhanced capacitive electrode operating in the low-coupling regime. The electrode dielectric was ordinary electrical-grade fiberglass circuit-board of thickness 0.74 mm.

FIG. 20 is a graph obtained in the chest belt configuration described in FIG. 16 but showing the signal approximately 5 minutes after placing ohmic electrodes made of conductive rubber directly on unprepared skin. For the first approximately 5 minutes no signal was seen.

FIG. 21 is a graph obtained in the chest belt configuration described in FIG. 16 but showing the differential, signal obtained using two enhanced ohmic electrodes. The electrodes were identical conductive rubber as before but with an enhancer layer constructed of a textile pouch filled with super-absorbent polymer granules fastened to the rubber. The enhancer was soaked with ordinary tap water and subsequently blotted with a towel.

FIG. 22 is a graph as in FIG. 21 but with the enhanced ohmic electrodes placed over a cotton T-shirt covering the skin of a human body. A signal, as depicted, very similar to that shown in FIG. 21 appeared approximately 2 minutes after placing electrodes on the clothing layer.

FIG. 23 is a graph as in FIG. 20 but with the unenhanced ohmic electrodes placed over a dry cotton T-shirt over the skin of a human body. No ECG signal was evident but only 60 Hz artifact and noise.

FIG. 24 is a graph as in FIG. 16 taken with a pair of enhanced capacitive electrodes with moistened enhancer placed over a hydrophobic polyester fabric layer over the skin of a human body. The settling of the signal can be seen as a result of the moisture penetration from the enhancer into the clothing fabric.

FIG. 25 is a graph as in FIG. 24 taken through a cotton fabric over the skin of a human body and showing a relatively rapid settling time.

FIG. 26 is a revised version of FIG. 1B wherein the presence of an enhancer layer, together with the skin and small air gaps are modelled by parasitic resistance Cₚₐ and resistance Rₚₐ in parallel.

### DESCRIPTION OF THE PREFERRED EMBODIMENT

While aspects of the invention are suited for use with conventional electrodes of both the ohmic and capacitive type, a special type of preferred electrode will first be described before addressing the electrode enhancer of the invention.

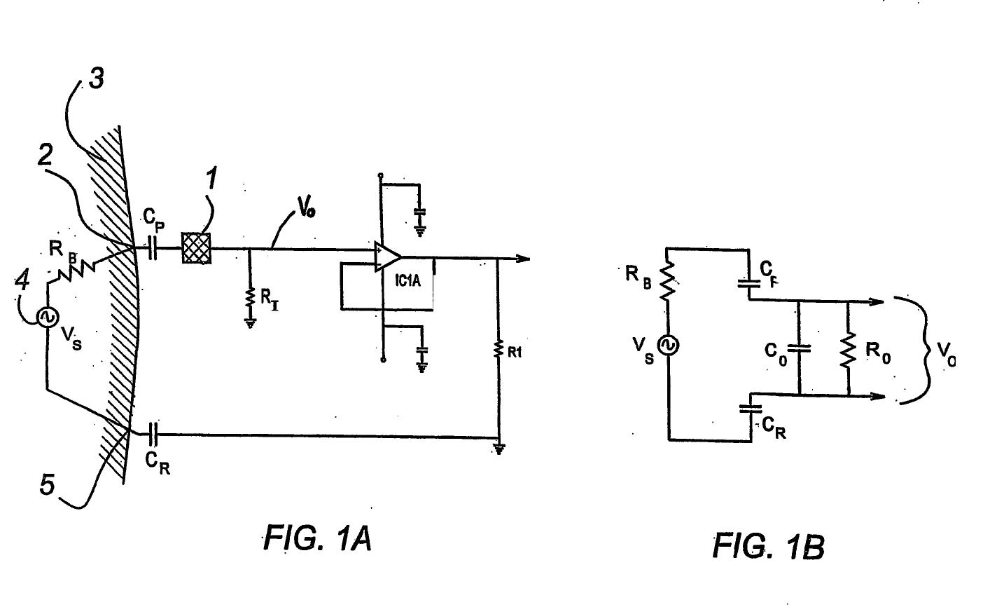

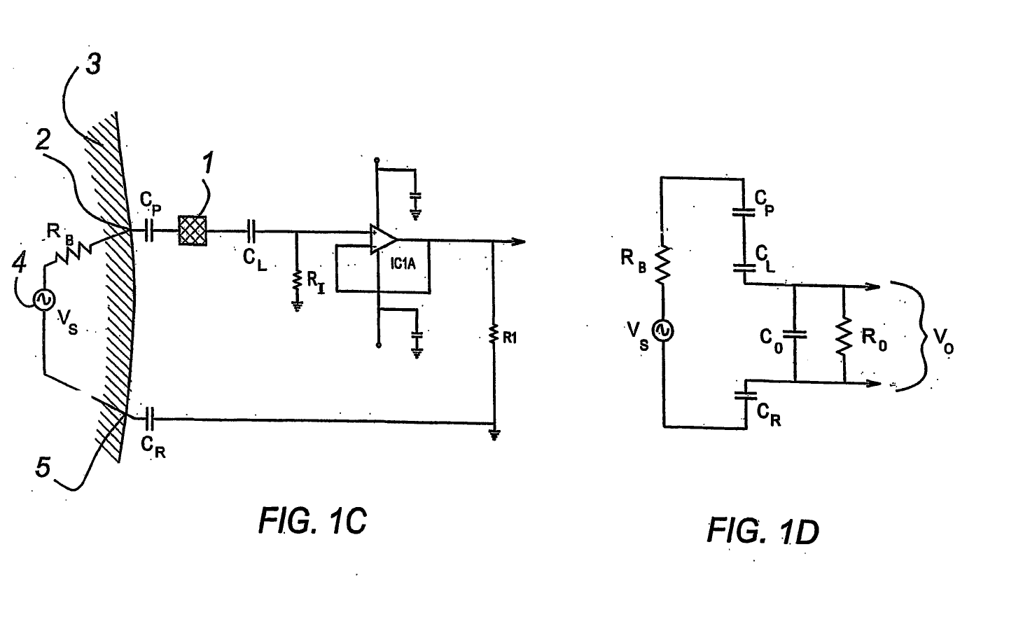

In FIG. 1A a pictorial schematic is shown of an electrical sensor system for a capacitive ECG sensor incorporating a pick-up electrode 1 in the form of a flat conductive surface placed adjacent a first location 2 on a body 3 where an electrical signal is to be sensed originating from an electrical signal generator 4 within the body 3 that provides a source voltage Vₛ. The pick-up electrode 1 develops a capacitive coupling to the body 3 through an intervening dielectric layer separating it from the body 3. This capacitive coupling for the pick-up electrode 1 is represented schematically by the capacitor Cp.

The electrode 1 is connected to the input of an operational amplifier—IC1A, or its equivalent such as a field effect transistor. Input resistance R_{I} connected between the amplifier input and circuit ground has a resistance value of on the order of 10¹² ohms and serves to discharge the input of DC offsets and restore proper voltage input levels while accepting signals of the desired frequency.

The output Vₒ from the voltage divider network which drives the operational amplifier IC1A is measured across input resistor R_{I} that extends between the input of the operational amplifier IC1A through circuit ground to a reference capacitor C_{R} that is coupled to the body 3 at a second, separate location 5. This location 5 may be separated from the first location 2 in obtaining conventional ECG signals. The locations 2,5 may also be proximate, e.g. adjacent, at certain body locations and still provide useful signals.

Capacitive coupling C_{R} is effected by means of an electrode (not shown in FIG. 1A) that is separated from the body 3 by a non-conducting material that acts as a dielectric. Conveniently, the case for an on-board battery holder can serve as this electrode, as shown further below.

The nature of the dielectric material has little effect on the pick-up obtained by capacitive electrodes operating in low-coupling regime and for electrodes of the present invention in which an enhancer prevents direct contact between the electrode dielectric and the body. In each case, electrodes can be placed in ‘casual’ mechanical contact with the body being sensed as in the case of ECG pick-up on hairy skin or over clothing. Satisfactory values of dielectric constant for the dielectric layers have been found in the range 1 to 10. Compared to prior art, a much greater variety of flexible, non-brittle dielectrics can therefore be used. A further advantage of low-coupling capacitive electrodes is that electrodes with thick dielectrics can be more robust compared to prior art thin-film dielectrics.

Inside the body 3, the signal generator 4 is seen as being subject to internal resistance R_{B} within the body 3.

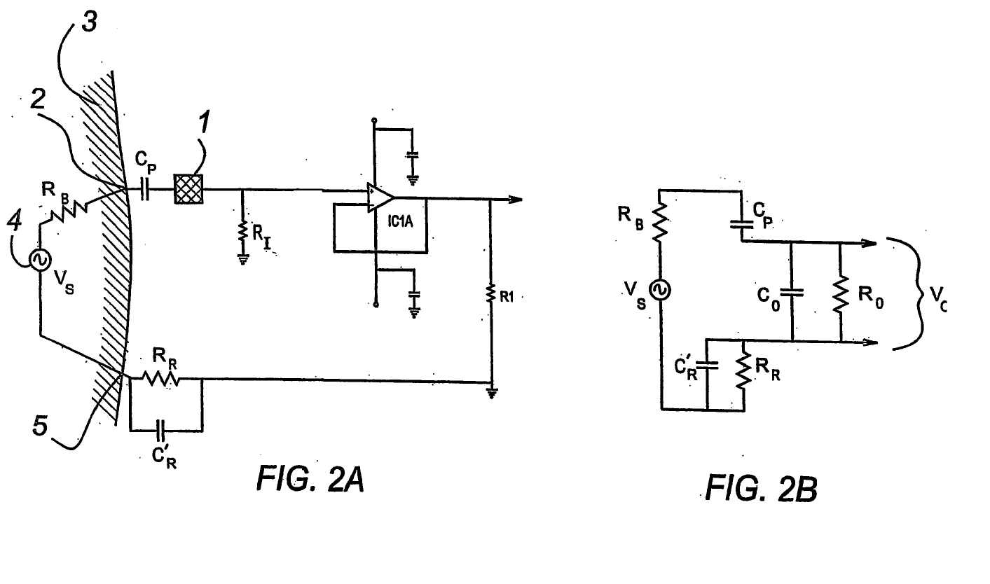

The input portion of circuit of FIG. 1A is redrawn as FIG. 1B in more conventional form, depicting a closed circuit voltage divider network. In FIG. 1B, the capacitance Cₒ arises from the combined input capacitance of the operational amplifier IC1A and the input resistor R_{I} and due to geometrical capacitances arising between the circuit, the electrode and the shielding elements. The total apparent input resistance of the amplifier is represented by Rₒ, including the resistive value of the input resistor R_{I}. Collectively, the capacitances Cₚ, Cₒ, C_{R} act as a voltage divider network whereby the output voltage Vₒ is proportional to the source voltage Vₛ. In FIGS. 2A and 2B, the coupling to the body 3 at the end of the voltage divider network opposite to the pick-up electrode 1, is effected principally by a direct, conductive contact. The resistance of the interface is indicated by R_{R}. Necessarily, some slight capacitance coupling is also still present, indicated by C′_{R}.

The output signal of the sensor is extracted by measuring the voltage difference across an electrical component in the voltage divider network that is connected to the subject electrical source. This should be done through a high impedance, low capacitance sensing circuit or sensing means to minimize signal loss. A field effect transistor or operational amplifier having an input impedance of on the order of 10¹⁴ ohms and an input capacitance of about 3 to 5 picofarads has been found to be satisfactory when the other capacitor(s) in the voltage divider network have values of on the order of 10 picoFarads. Signals can be obtained with a circuit signal sensing means having an effective input capacitance of on the order of 15 to 20 or 25 picofarads. The lower the input capacitance, the less signal loss occurs. It is preferable that the electronic circuit sensing means have an input capacitance of less than about 25 picofarads, preferably less that 15 picofarads, more preferably less than 10 picofarads. Used in conjunction with a pick-up electrode having an area of on the order of one to ten square centimeters, dielectric media having a total effective dielectric constant of 1-10 and a body-to-surface gap distance of on the order of 0.1 to 4 millimeters, signal values of the order of 1 millivolt or less may be detected from the skin surface of the human body.

With this type of sensor configuration useful signals may be obtained with the plate of the pick-up electrode separated from the skin or sensed body by a gap that allows the pick-up to qualify as a “low-coupling” electrode. By operating the sensor in the capacitance/gap separation region specified hereafter, variations in skin properties, sweating, body motions, will not detract inordinately from the signals being obtained.

A pickup electrode that is removed somewhat from the electrical field source is able to supply a satisfactory signal by reason of the mathematical relationship that exists between the value of capacitance and the separation distance existing between capacitor plates or electrodes. Since capacitance varies inversely with separation, the mathematical form of a curve for capacitance value plotted against separation distance is in the shape of a hyperbola. This means that the capacitance performance of a pickup electrode can operate in two distinct regions:

1) a first region wherein the separation distance is small and the curve is steep, corresponding to the situation in the prior art where the capacitance value will vary highly, with great sensitivity, in response to small changes in the separation distance; and

2) a second region wherein the separation is greater, the curve is relatively flat, and the capacitance value varies relatively insensitively with similar changes in the separation distance or body surface layer. For the purposes of the present invention, the preferred region of operation according to one variant of the invention is in the second, separation-insensitive zone.

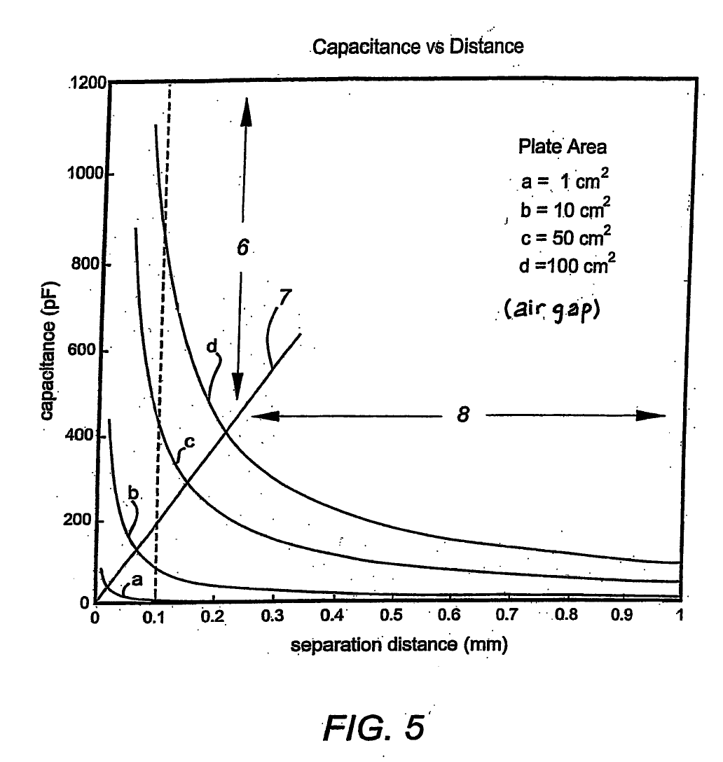

In FIG. 5 a graphic plot is depicted of the variation of capacitance C with a variation in the separation distance d at various separation distances d, based upon the theoretical formula:

C=e.A/d

where: C is the effective capacitance of, for example Cₚ,

d is the separation distance of the electrode plate from the body giving rise to the capacitance,

A is the area, or effective area, of the pick-up electrode 1; and

e is a proportionality constant determined by the dielectric constant of the material in the separation gap. In FIG. 5 the value of the dielectric constant is assumed to be that of air, i.e. 1.0 and the plates forming the capacitance are assumed to be fully conductive. This is therefore an idealized variant on the case of coupling to the human body.

Four curves are shown in FIG. 5 for pick-up electrodes 1 having surface areas as follows:

| | |
|---|---|
| a = 1 cm² | c = 50 cm² |
| b = 10 cm² | d = 100 cm² |

Each capacitance curve can be separated into two important regions: region 6, in which the capacitance changes relatively rapidly with a given change in separation distance; and region 8 in which the capacitance changes relatively slowly with a similar given change in separation distance. These regions are generally separated on FIG. 5 by boundary line 7. For a capacitor with an electrode area of 1 cm², the line 7 passes approximately through a capacitive value of about 40 picoFarads. For capacitors with an electrode area of around 25 cm² and capacitive values below 200 picoFarads, region 6 approximately corresponds to the zone with d=0.1 mm or less; while for such values region 8 approximately corresponds to the values above d=0.1 mm. Note that 0.1 mm is approximately the thickness of human hair and can be considered as a separation gap typical of parasitic capacitance on hairy skin.

A preferred criterion for the operation of a capacitor in the low-coupling position-insensitive region is that a 0.1 mm increase in the capacitor-to-body gap causes a 50% or less change in the net capacitance of the electrode to body coupling. A rate of change of capacitance Cₚ with variations in the position of the electrode equivalent to this criterion essentially characterizes region 8.

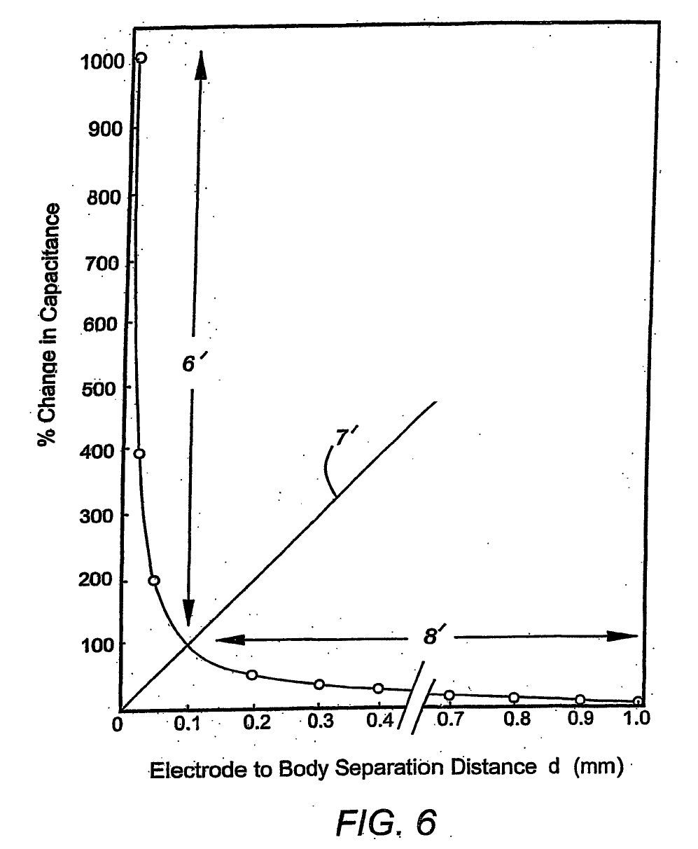

An important implication of FIG. 5 is that sensors designed to operate with capacitance values within regime 6 require a high degree of intimacy of coupling to body and are very sensitive to small additional changes in the separation distance (delta-d). Such electrodes are also sensitive to changes in the electrical properties of the skin and its parasitic capacitance as may arise, for example, through variations in the amount of sweat present. In contrast, sensor systems with capacitance values corresponding to region 8 and corresponding electronics are relatively insensitive to body coupling and to such changes. This is illustrated more succinctly in FIG. 6.

These factors can be conveniently summarized by way of considering the sensitivity of the electrode coupling value to small changes in separation distance d. In FIG. 6, the percentage change in capacitance corresponding to a delta-d=0.1 mm is graphed as a function of the nominal separation distance d.

FIG. 6 is dimensionless along the C axis and applies to all capacitive sensors which obey or approximately obey the relation C=eA/d. A preferred application of enhancement layers according to the invention is based on capacitive sensors which when employed, are designed to operate in region 8′ of FIG. 6, as opposed to region 6′ from which it is separated by boundary line 7′. In this former regime 8′ the capacitance, and hence the output signal is sufficiently insensitive to spatial body surface variations so as to contribute to the advantages of signal stability.

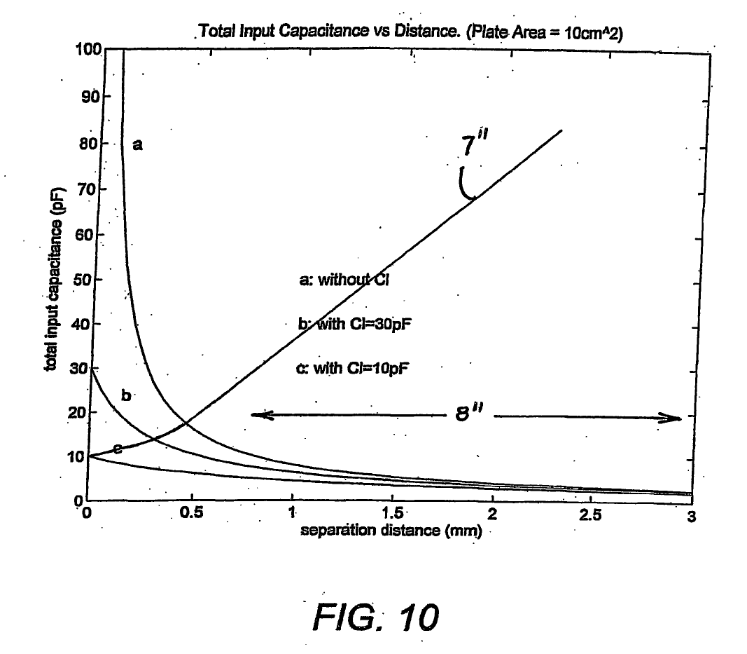

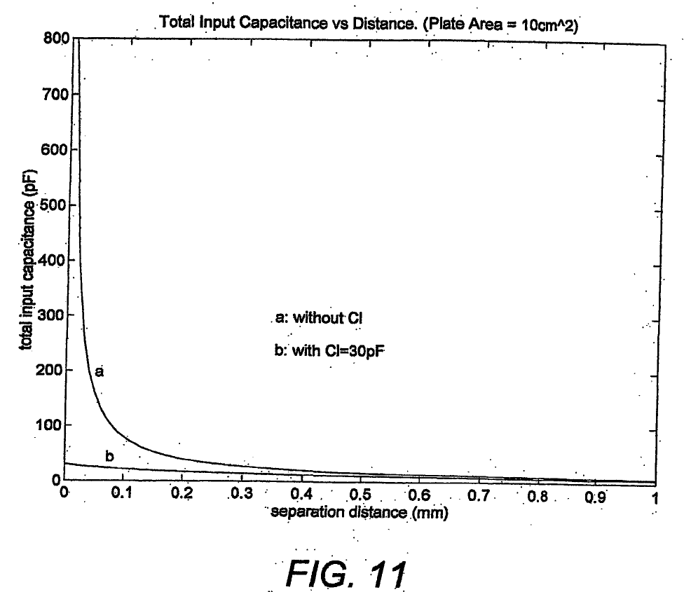

FIGS. 5 and 6 premise that operation in regions 6 and 6′ can be effected by achieving, or tolerating, low capacitance coupling between the body and the pickup electrode. FIGS. 10 and 11 apply to an alternate case wherein the capacitive coupling between the pickup electrode and the body is high, but the results of achieving system operation in preferred regions 6,6′ is still obtained. This is achieved by insertion of a series limiting capacitor C_{L} in the input to the first stage amplifier of the sensor.

This series limiting capacitor may have a preferred value that is greater than the input capacitance of the first stage amplifier, and less than the effective value of the capacitance coupling between the pickup electrode and the body whose electrical field is being sensed, e.g. between 5 and 40 picoFarads.

In FIGS. 1A and 1B the pickup capacitor Cₚ is shown as being directly coupled to the operational amplifier 1C1A. In FIGS. 1C and 1D a series capacitor C_{L} is shown added between the pickup capacitor Cₚ and the amplifier input (at which Vₒ is detected). The effect of this limiting capacitor C_{L} is to place a maximum value on the capacitance extending between the body 3 and the signal sensing means C1A. The pickup electrode's capacitance Cₚ is in series with the limiting capacitor C_{L}. Collectively, they behave as a single capacitor having a total net value C_{T=l/(l/C}_{L+l/C}ₚ).

FIGS. 10 and 11 plot the behaviour of C_{T} as a function of the separation distance present for the pickup capacitor Cₚ. This net value capacitor C_{T} provides a more stable, separation-insensitive circuit performance that occurs in its absence. This is particularly true when C_{L} is smaller than Cₚ.

A convenient formula for establishing a value for C_{L} is that C_{L} should be less than 5 (picoFarads/cm²) times the area of the pickup electrode (in cm²).

The consequence is that a similar region 8″ of insensitivity to displacement of the pickup electrode exists in FIGS. 10 and 11, parallelling regions 8 and 8′ in FIGS. 5 and 6. A similar preferred criterion for performance of a capacitor in the position-insensitive region can also be established for the circuit arrangement of FIG. 1C, 1D, namely, the rate of change of capacitive Cₙ with variations in the position of the electrode is equivalent to a 0.1 mm displacement of the pickup electrode causing a 50% or smaller change in the net capacitance Cn. Preferably the change is less than 20%.

Thus the effect of desensitizing the signal pickup and coupling capacitance can sometimes be achieved through the presence of a limiting capacitor C_{L} in the input link between the pickup capacitor Cₚ and the signal sensing means 1C1A.

For the present invention, the input resistance present at the input to the high impedance amplifier can be provided from two sources:

1) the inherent input resistance of the amplifier, typically 10¹³-10¹⁴ ohms;

2) the input resistance of an added, external, input resistor, R_{I} between input and reference voltage.

A preferred value for this resistance R_{I} may be determined by considering the pickup electrode and input resistance as an RC high frequency passing filter.

Assuming an effective pickup electrode capacitive value of 60 picoFarads and a low frequence cut-off of 0.05 Hz established by the RC input value of the first stage amplifier, a preferred value of 4×10¹² ohms may be provided for the input stage input resistance R_{I}.

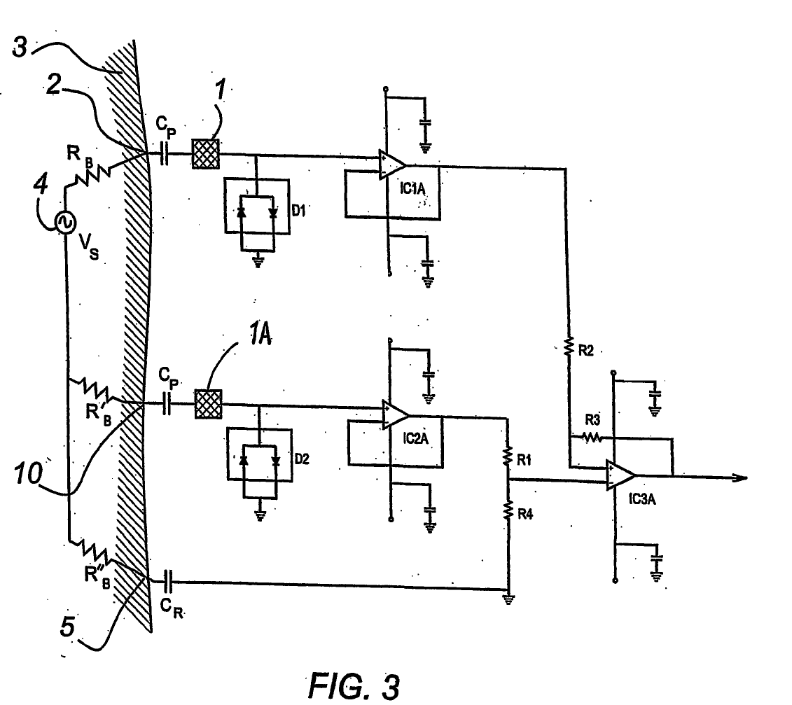

Occasionally, the near-DC signals delivered to the pickup electrode will be so substantial as to drive the signal at the input amplifier to the limit of its range of response. When overdriven, the recovery period before a normal input level is established can be shortened by providing a special input resistor arrangement at the amplifier input. In such cases it is convenient to provide the input stage with a non-linear input resistance. This can be achieved by grounding the input through pairs of Schotkey diodes, D₁, D₂ in FIG. 3, connected in parallel.

The forward resistance of Schotkey diodes before breakdown occurs can be on the order of 10¹³ ohms. By choosing diodes with a forward breakdown voltage that is above the level of the signal of interest, the “reset” function of the input resistance of the high impedance amplifier can be improved.

As the resistance of the Schotkey diodes prior to breakdown may be higher than the appropriate value to provide an input resistance suited to the given low frequency cut-off for the RC filter, such diodes D_{1,} D₂ may have to be accompanied by a parallel input resistor R_{I} that establishes the appropriate net value for input resistance for small level signals.

In FIG. 3 two pick-ups similar to that of FIG. 1A (except for the substitution of diodes D₁, D₂ for the input resistor R_{I}) are used to drive a differential amplifier IC3A. The second additional pickup electrode 1A is placed at a location 10, separated from the first and second locations 2 and 5. Within the body the signal source Vₛ may be treated as distributing its potential over the resistors R_{B}, R′_{B}, R″_{B}.

By use of this differential signal detection circuit, common mode noise present in the two pick-up circuits will be minimized. In some cases the connection to location 5 through C_{R} may be omitted as a signal can be obtained from locations 2 and 10 only.

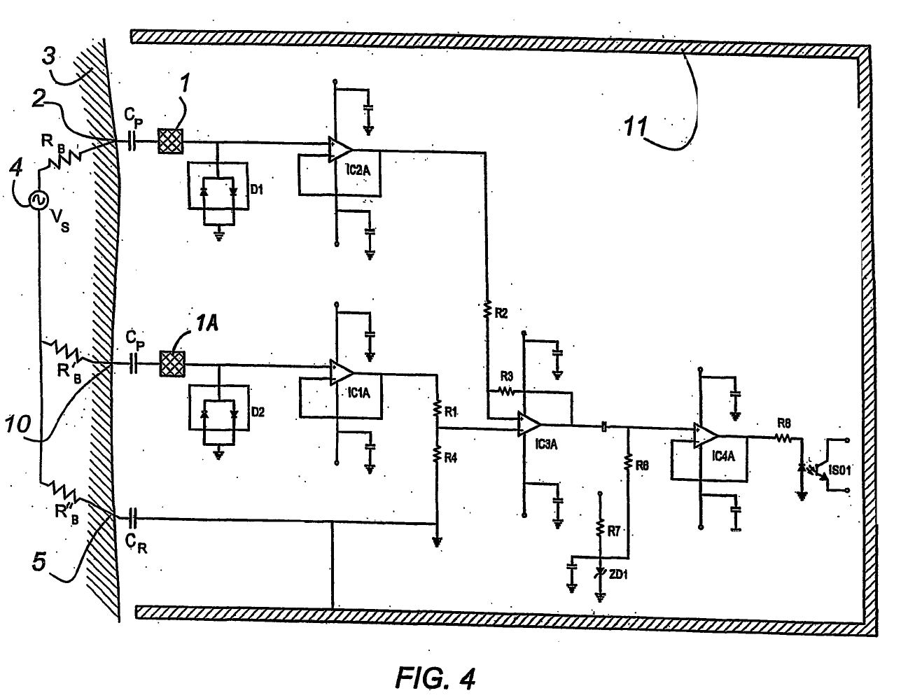

FIG. 4 shows the circuit of FIG. 3 extended by an optical isolator IS01 driven by an operational amplifier IC4A which is, in turn, driven by the output from the differential amplifier 1C3A. By mounting these circuits as close as possible to the pick-up electrodes 1 1A, interference from ambient 60 Hz electromagnetic signals can be minimized.

In FIG. 4, a shielding conductive layer 11 is depicted as overlying the externally-directed side of the circuitry. This layer/structure 11 is preferably connected to the circuit common point but need not necessarily be so connected. In some configurations this shield may be “floating”. Its role is to exclude effects arising from intruding electromagnetic signals, e.g. 60 Hz, originating in the environment. In non-earthed applications the shield distributes ambient, intruding signals equally to both pickups thus contributing to common mode noise rejection. It is highly desirable that such a shield be employed in one or other of such configurations.

The “low-coupling” capacitive electrode as described has advantages over conventional ohmic and capacitive electrodes in that it need not be intimately pressed or adhered against the body which is the source of the field being sensed. In fact, useful signals can be obtained with a loosely positioned pickup electrode, and even through some types of fabric. This opens-up possibilities for the long term monitoring of patient heart rate and ECGs without the use of uncomfortable adhesives found in typical existing ohmic electrodes.

While useful signals can be obtained directly through certain types of fabric, e.g. cotton, by using a low-coupling electrode according to the invention, noise has been found to be present in a variety of differing fabrics and environmental conditions. Noise is believed to originate primarily from tribo-electric effects that arise when surfaces touch or slide with respect to each other. A feature of the present invention is the inclusion of an “enhancer” layer between the electrode and the body that reduces the amount of noise occurring in respect of the sensed signal.

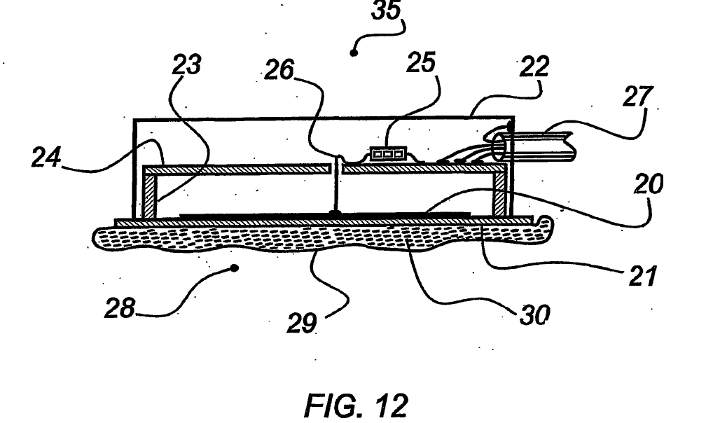

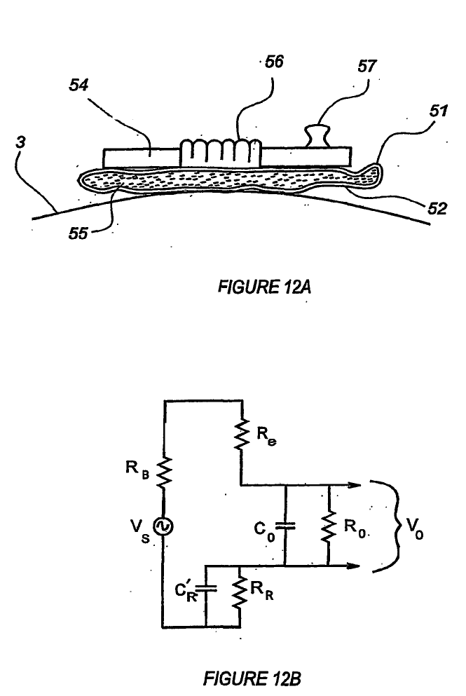

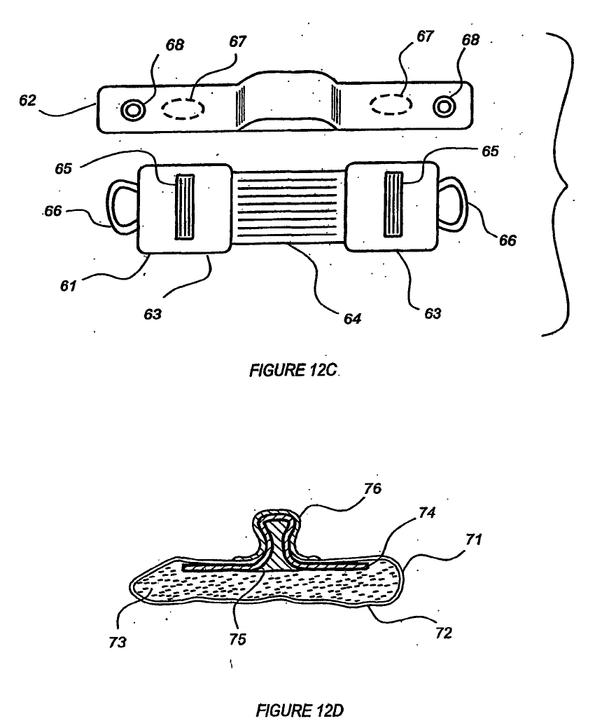

In FIG. 12 an electrode 35 with on-board electronics has a conductive plate 20 covered by an insulative dielectric layer 21 and preferably encased with a shielding cap 22. To support the electronics, a circular ring 23 supports a board 24 that carries a high impedance input amplifier circuit 25. A lead wire 26 connects this circuit 25 to the plate 20. Output wires 27 carry the output from the circuit 25 to further circuitry (not shown).

Beneath the dielectric layer 21 is positioned an enhancer 28. The enhancer 28 is preferably positioned adjacent to the dielectric layer 21; more preferably it is bonded to the dielectric layer 21, as by an adhesive. In the case of an ohmic electrode the enhancer 28 may be adjacent to or bonded to the surface of the ohmic electrode, as by a conductive adhesive.

The enhancer 28 has a body-facing surface 29 which maybe a textile, permeable polymeric sheet or the like that acts as a containment layer and provides a relatively non-slippery engagement to occur between the enhancer 28 and a body 3. Cotton textile has been found to be suitable for this purpose. The enhancer 28 may optionally have a cushioning volume of material 30 present between the facing surface and the dielectric layer 21. This material 30 is preferably pliable or conformable, sufficient to cushion the electrode 25 against small displacements of the body 3 with respect to the electrode 35. Alternately, the textile itself may perform this function.

A preferred cushioning material is a granular assembly of super-absorbent polymer, e.g. cross-linked sodium or potassium polyacrylate/polyacrylamide (CLP). A desirable characteristic of CLP which is a preferred feature of the cushioning material 30, is that it has the capacity to both contain and emit water. Further CLP, as a preferred characteristic for the cushioning material 30 is relatively conductive of electricity when moist, operating by ionic conduction.

When placed against skin of a body 3, CLP as the cushioning material 30 will evolve or emanate water by diffusion which will introduce moisture into the electrode-to-body interface. Water transferred from the CLP to the interface will be slightly ionically conductive as a result of contact with the CLP and with skin or clothing. Alternately or additionally, sweat will provide salt to the interface, thus raising the electrical conductivity of the skin-to-electrode interface.

FIG. 12a illustrates an enhancer 51 having a flexible, moisture-permeable containment means 52 enclosing and containing a super-absorbent polymer material 55, preferably CLP in granular form. The electrode 54 whose performance is enhanced by the presence of the enhancer 51 is an autonomous, pre-existing electrode of the capacitive or ohmic type. The hydrated enhancer 51 is placed between the electrode 54 and the body 3 and is held in position via a strap 56 or via any external means that connects the electrode 54 or the electrode output connector 57 to the sensing device.

The containment means 52 is permeable to moisture and is preferably flexible, compliant and bag-like. The enhancer 51 is hydrated by wetting with ordinary tap water whereupon the super-absorbent material 55 contained therein absorbs water and swells in volume. The enhancer 51 then becomes soft, pliable, cushion-like and moisture emanating.

The moisture-permeability of the containment material 52 should allow an effective amount of moisture to be released from the enhancer 51 so as to provide an improved electrical coupling between electrode 54 and body 3 while at the same time not releasing an excessive amount of moisture to avoid discomfort to the user or the rapid depletion the moisture contained by the enhancer 51. In the case of enhancers constructed with containment layers of ordinary textile fabrics and containing superabsorbents consisting of CLP granules of size 100 microns or less, it has sometimes been found advantageous to provide two layers of textile as the containment 2 in order to reduce the moisture-release rate from the enhancer, particularly when finer CLP granules are used. In the case of granular superabsorbents it has been found that the wetness or rate of moisture release from the enhancer is a function of the superabsorbent granule size and the containment layer porosity and thickness. Enhancers that feel dry to the touch are achieved by using large superabsorbent granules and small containment layer porosities or thick containment layers of fixed porosity. Enhancers that feel wetter are achieved by using finer superabsorbent granules with thinner containment layers or more highly porous containment layers, the limit to wetness being determined by the requirement that the containment layer retains the ability to resist permeation by the finer superabsorbent granules themselves.

The containment layer 52 should be microporous and preferably sheet-like. Materials found suitable for the containment layer 52 are ordinary textile fabrics, non-woven fabrics, cellulose mat, and synthetic open-cell foam or sponge materials which are available in sheets. It is preferable for the material to be heat seal-able thereby simplifying the manufacturing of the containment structure.

To prevent odors caused by the growth of molds, bacteria or other microorganisms, the CLP or the containment means 52 can be treated with any of a number of common antiseptic or bacteriocidal chemicals used in cosmetics and medicine. An enhancer 51 of this type can be reused many times, and can be re-hydrated by immersion in tap water through many cycles of wetting and drying.

FIG. 12b illustrates the equivalent electrical circuit for the ohmic electrode 54 and enhancer 51 of FIG. 12a. This is similar to FIG. 1 for capacitive electrodes, but with a resistor Re in the place of the capacitor Cp seen in that figure. In FIG. 12b Re represents the electrode-to-body total resistance and is equal to the series sum of the electrode resistance and the enhancer resistance.

FIG. 12c illustrates a pre-formatted enhancer array or module 61 designed to be fitted and enhance the performance of commercially available heart rate transmitter belt 62 used to monitor a user's heart rate during exercise. The enhancer module 61 consists of two enhancers 63, of the fabric pouch type illustrated in FIG. 12a. The enhancers 63 are each of a size that matches the commercial heart rate belt 62 existing electrodes 67. The enhancers 63 are spaced via a central strap 64 which facilitates positioning of the enhancers 63 under the commercial heart rate belt 62 electrodes 67 when the commercial belt 62 is applied to the body 3 with the enhancer assembly 61 intervening between the commercial belt 62 and the body 3. The central strap 64 is preferably of low electrical conductivity so as to prevent signal shunting between the enhancers. Ordinary hydrophobic fabrics have been found suitable for this purpose. Small vertical loop straps 65 are provided towards the outer ends of each enhancer 63 to further facilitate placement of the enhancer assembly under the commercial heart rate belt 62 and to prevent slippage and relative movement between the enhancer array 61 and the commercial electrode assembly 62 it enhances. Optional end loops 66, as with the openings 68 in the belt 62, are provided to enable the enhancer array 61 to be strapped to the user's chest using elastic strapping means provided with most commercial heart-rate monitor belts.

FIG. 12d shows in cross-sectional view an enhancer 71 with an electrode 74 incorporated within it. The enhancer 71 consists of a water permeable bag or containment means 72 containing super-absorbent polymer 73. The electrode 74 is a conductive sheet or layer, preferably compliant, which is positioned inside the bag 72. Suitable materials for electrode 74 are metallized conductive fabrics, conductive plastic films, conductive elastomeric polymers, or metal buttons that may be chloridated as in conventional ECG electrodes. In the case of metal or metallized electrodes 74, the metal used should be selected so as to be compatible with the super-absorbent material 73 i.e. such that it does not significantly degrade the super-absorbent polymer 73 when wetted. For CLP-based superabsorbents 73 nickel plated fabrics have been found preferable to copper. Also suitable for this purpose is carbon-impregnated conductive rubber.

Electrical connection to the electrode 74 of FIG. 12d can be accomplished by a crimped connector consisting of upper 76 and lower 75 mating parts that interpenetrate each other. When crimped through the upper layer of the containment means 72 and the electrode 74 layer this connector ensures ohmic contact between the outer portion of the connector 76 and the electrode 74. Standard clothing dome connectors 76 have been found suitable for this purpose as they are low cost and possess the advantage of connecting to female snap connectors customarily used for heart rate and ECG pickup devices.

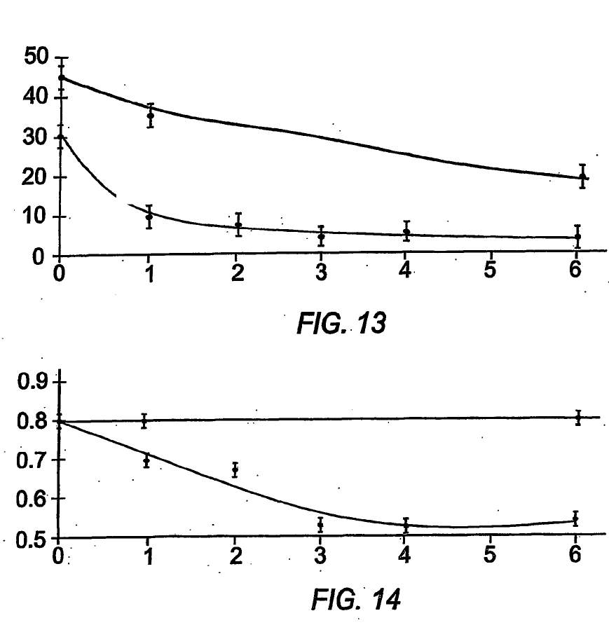

FIG. 13 is an illustrative graph showing the general trend for the approximate signal to noise ratio (SNR) for a signal obtained by an enhanced capacitive electrode over multiple layers of dry T-shirt fabric on the body. The layers were obtained by upwardly folding the T-shirt around the chest. The electrodes were held in position several centimeters below each nipple of the test subject by use of an elasticized fabric strap placed over the clothing fabric around the chest. Electrodes were connected to the lead-one inputs of a commercial ECG machine. All other ECG inputs were grounded to the subject body-reference via a stainless steel plate at the right foot.

The SNR was estimated by measuring average peak-to-peak voltages of the cardiac ventricular depolarization peak and the noise within the band 0.5 Hz to 30 Hz. The enhancer 30 is based on salted cotton denim adhered to the electrode dielectric. The signal to noise ratio is plotted against the number of layers of cotton T-shirt fabric, both for a water-moistened enhancer and for an ostensibly dry enhancer layer. In all cases the moistened enhancer provides a higher signal-to-noise. The SNR declines more rapidly for the ostensibly dry enhancer than for moistened enhancer as the number of layers of clothing fabric are increased.

FIG. 14 is an illustrative graph based on the same measurement series as FIG. 13, showing the general trend for the signal strength of the ventricular depolarization “R” peak as a function of the number of layers of cotton T-shirt between the electrode of FIG. 13 and skin. In this case, the moistened enhancer shows relatively little signal loss with an increase in the number of clothing layers while the ostensibly dry enhancer provides a reducing signal strength as the layers increase. This illustrates signal loss due to parasitic capacitance of clothing for a capacitive electrode operating in the low-coupling regime.

FIG. 15 is a dual curve graph showing ECG signals obtained directly over unprepared skin for a pair of unenhanced capacitive electrodes, curve 50, and a pair of prior art ‘wet-gel’ ohmic pickup electrodes produced by the 3M company of Minnesota, curve 51. One electrode from each pair were positioned side by side on the body at approximate MCL3 locations. Each electrode pair provided signals to nominally identical electronic circuitry in differential mode corresponding to FIG. 3, but with the high-impedance input stage bypassed for the ohmic electrodes. The capacitive electrode pickup represents an acceptable ECG signal although the capacitive electrode signal amplitude is only about 60% of the ohmic electrode signal amplitude for the particular devices used.

FIG. 16 depicts the ECG graph obtained on skin with a capacitive electrode possessing a moist, salted cotton denim enhancer layer. The signal was stable and 100% of the signal amplitude predicted on the basis of the electrode nominal capacitive coupling.

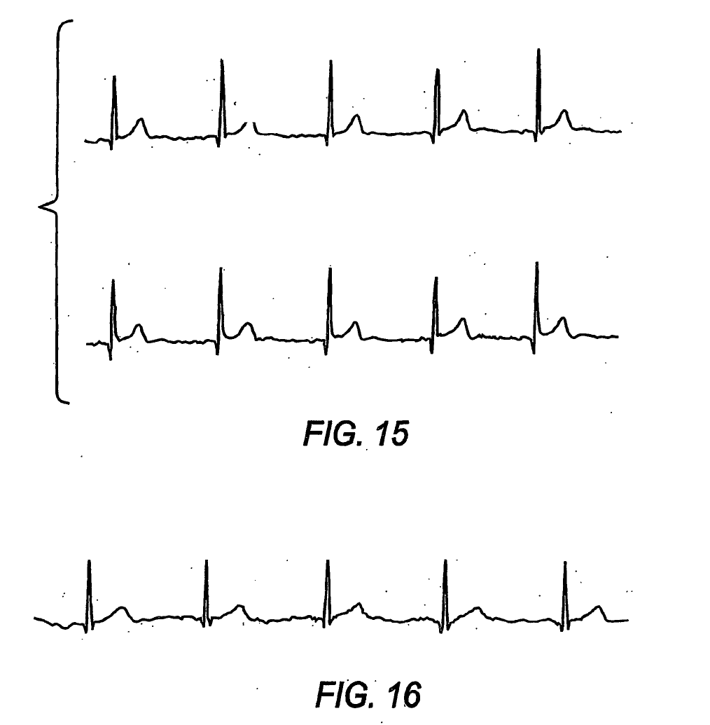

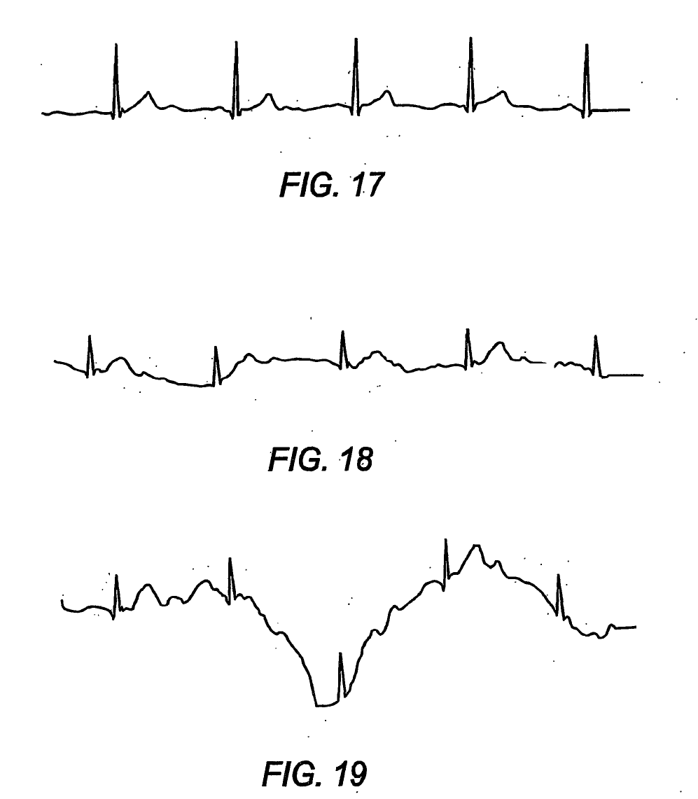

FIG. 17 is an ECG graph obtained with the enhanced capacitive electrode of FIG. 16, moistened and applied over a single layer of a coarse woven cotton golf shirt. This signal is essentially the same as obtained directly over skin in FIG. 16.

FIG. 18 is a graph as FIG. 17 wherein the enhancer layer has not been moistened. A loss of signal amplitude and an increase in noise is apparent.

FIG. 19 is a graph as in FIG. 17 but with no enhancer on the capacitive electrode. Without the enhancer layer there is a further reduction of signal amplitude and an increase in noise including significant baseline instability.

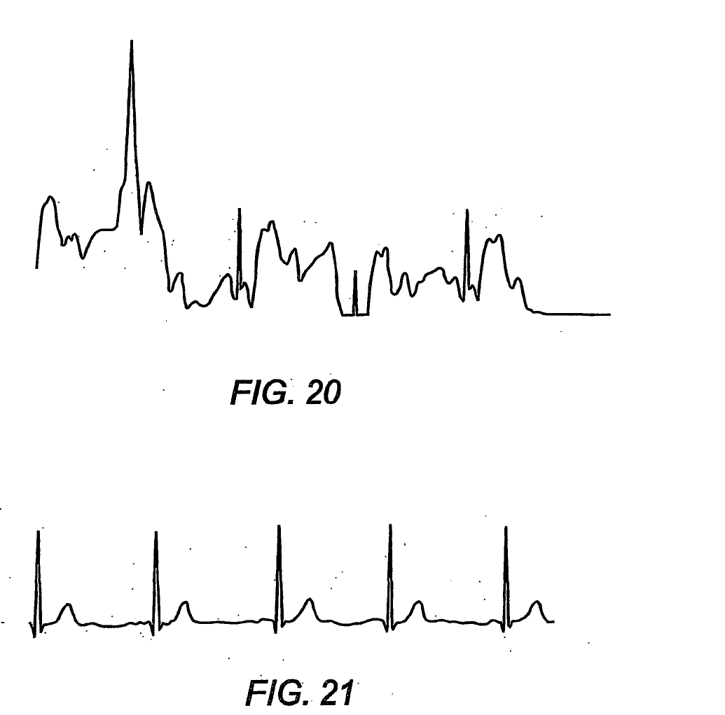

FIG. 20 is a graph of the ECG signal obtained with a conductive rubber ohmic electrode applied directly on unprepared ostensibly dry skin after a 5 minute delay. Initially there was no signal. This electrode is a prior art pickup used for trans-cutaneous electrical nerve stimulation (T.E.N.S.) and neural muscular electro-stimulation (N.M.E.S.) with its gel layer removed. The signal is unusable.

FIG. 21 is a graph of an ECG signal obtained with an enhancer layer present over the electrode of FIG. 20 as applied directly to the skin. The enhancer was a fabric pouch of cotton-polyester textile containing potassium based super-absorbent copolymer CLP granules which had been thoroughly wetted and then wiped surface dry. The signal was fully usable for ECG purposes.

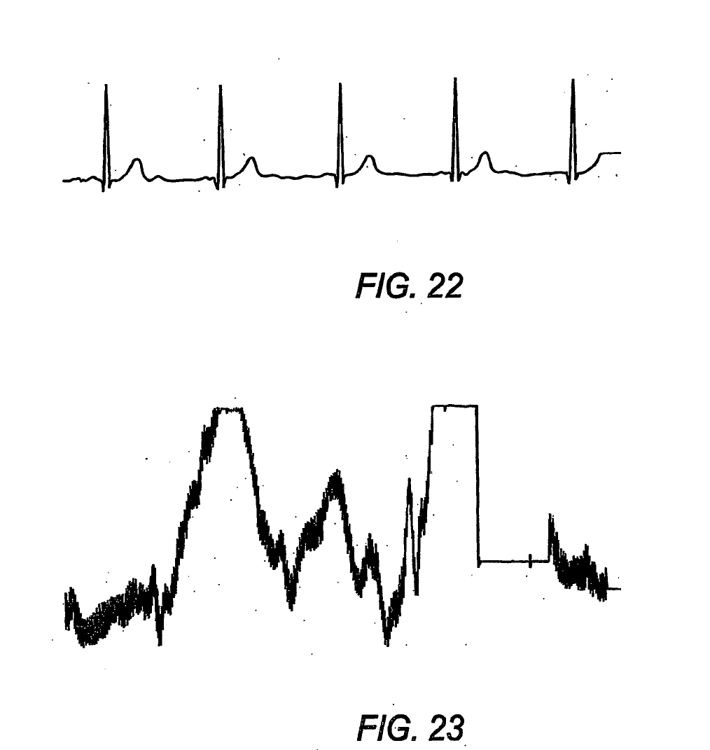

FIG. 22 is a graph as in FIG. 21 with the signal picked-up through a single layer of cotton T-shirt after allowing a minimum of 2 minutes for the signal to stabilize. The graph shown is taken after approximately 10 minutes. As of 2 minutes, the signal was equivalent in amplitude and morphology to its final form as in FIG. 22, except that it was more sensitive to motion artifacts. Such artifacts disappeared after 5-6 minutes. On removal of the electrode the T-shirt fabric previously in contact with the electrode was not soaked with water but was cool and lightly damp.

FIG. 23 is a graph of the signal obtained using an un-enhanced ohmic electrode as in FIG. 20 applied to a body through a layer of the same cotton T-shirt fabric as utilized in FIG. 22. No heart signal is present. Rather 60 Hz and baseline noise dominates.

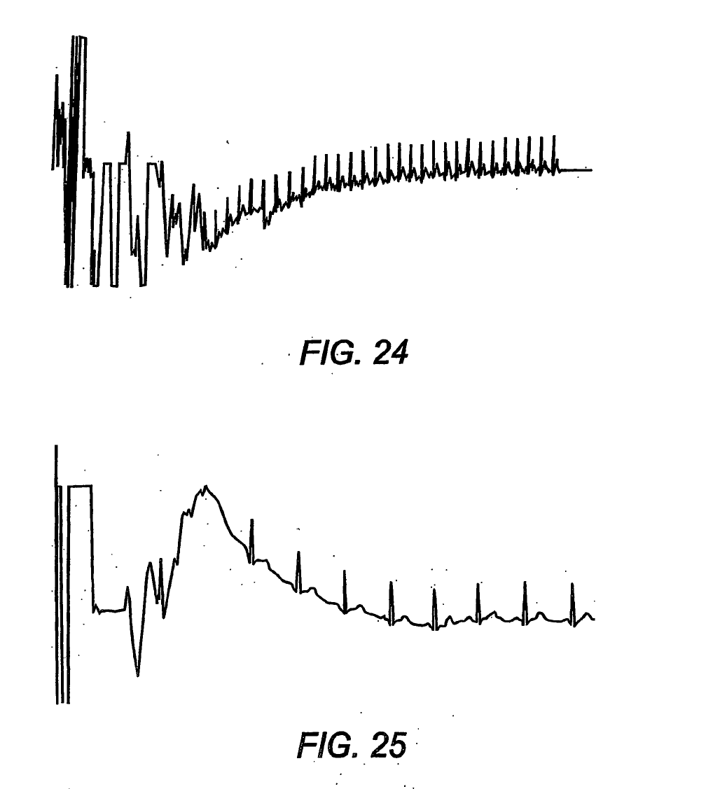

FIG. 24 is a graph of an ECG curve obtained through polyester fabric showing the relative stabilization of the signal with time arising from moisture from a moisture-carrying and emanating cotton denim enhancer layer providing moisture to the body-to-electrode interface. As moisture builds up in the polyester fabric it contributes to the quenching of interface noise. No noticeable wet spot was visible on the fabric after 30 seconds, by which time stabilization of the signal had occurred. Only a cool, moist region was formed under the electrode. This signal was obtained between unprepared skin on the right forearm and the clothing-covered precordial V4 region through a differential pickup circuit of the type of FIG. 3. Electrodes were held in position with elastic straps. A Burdick limb plate was used on the left wrist for body referencing. Ambient humidity was 34% and the temperature was 24° C.

FIG. 25 is a graph similar to FIG. 24 for a signal taken through a 100% cotton T-shirt fabric. Again, the signal settles with time as moisture rapidly penetrates the fabric of the T-shirt.

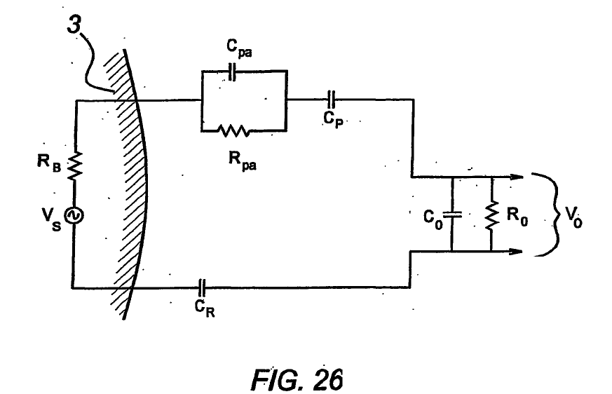

FIG. 26 is a revised version of FIG. 1B wherein the presence of the enhancer layer together with the horney skin layer (stratum corneum) and air gaps due to hair, etc. are modelled by parasitic resistance Rₚₐ and capacitance Cₚₐ in parallel. Through use of a conductive cushioning material with moisture-emanating properties such as CLP, values for Rₚₐ can be reduced to the order of several Megohms or less. The capacitance value Cₚₐ, while considerably larger than Cₚ becomes substantially irrelevant as a factor affecting signal pickup, once Rₚₐ stabilizes at its minimum value through moistening of the skin.

It is preferable in order to minimize noise for signals in the range 0.05 Hz to 100 Hz that the time constant of Rₚₐ and Cₚ be less than approximately 100 microseconds. A small electrode 35 such as shown in FIG. 12 has been constructed with a nominal Cₚ of 28 picoFarads as measured with the electrode 35 placed over a copper plate. Values for Cₚₐ typically range over 10-100 picofarads. With values for Rₚₐ on the order of or below 10 megohm, the output signal Vₒ is relatively independent of frequency in the ECG band of 0.05 Hz-100 Hz.

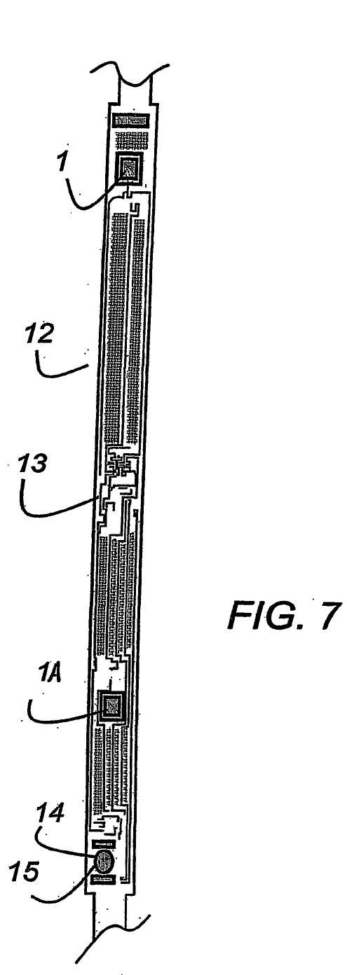

In FIG. 7 a belt 12 is depicted that carries the circuit of FIG. 4. The hatched areas are decorative. The pick-up electrodes 1, 1A are mounted on a KAPTON(™) film 13 that serves both as a spacer and as an insulating dielectric of approximately 0.13 mm thickness. The pick-up electrodes 1, 1A have been measured against a copper plate as providing a nominal capacitance value of 20 picoFarads.

The belt 12 of FIG. 7 has its own on-board power supply in the form of batteries 14. The case 15 of the batteries 14 is connected to circuit commons which together constitute a ‘floating’ ground network. When placed on the skin a capacitive coupling arises between the ground network and the body thus providing the reference capacitor C_{R}. A measured value for C_{R} when placed against a copper plate of 160 picoparads has been observed with the case 15 coupled to the entire circuit. C_{R} is not essential for differential pickup.

The substrate for the belt 12 is made of KAPTON (™) having a thickness of 5 thousandths of an inch. This forms the principal dielectric element for both of the capacitors Cₚ and C_{R}. The nature of the dielectric material has little effect on the invention when the pick-up electrode plates are located at a sufficient separation gap from the body as when an enhancer layer is present.

The shield 11 (not shown) in the belt 12 of FIG. 7 is in the form of a flexible conductive layer, with an insulated undersurface that overlies the circuitry on the outer side portion of the belt 12. This shielding layer must be close enough to the pickup electrodes 1 to evenly distribute ambient noise signals, and be sufficiently spaced from the pickup electrode/body interface so as to not detract from signal pickup by the pickup electrodes.

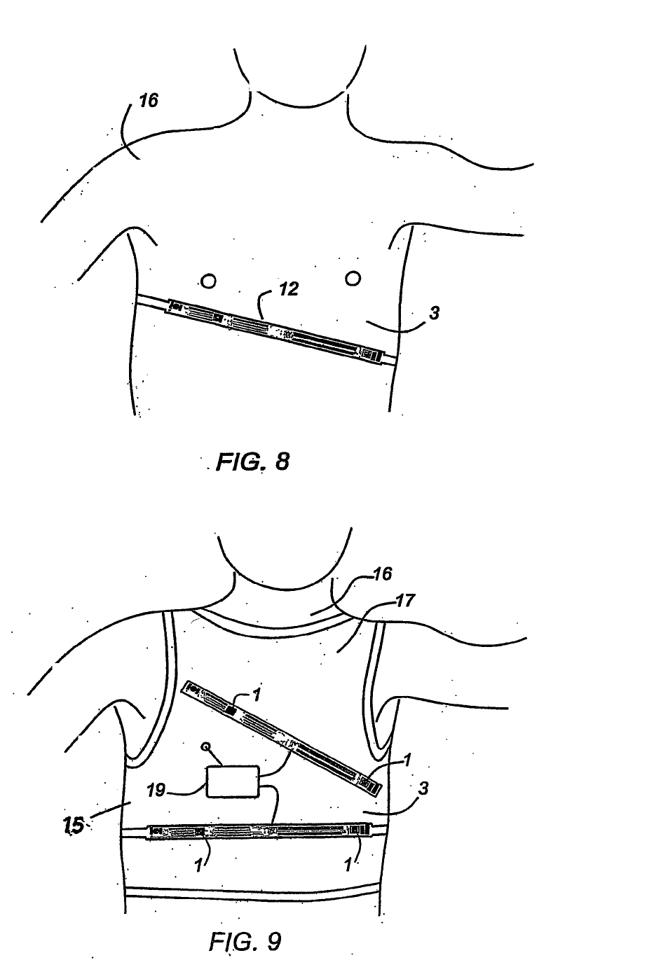

The pick-up electrodes l, 1A in FIG. 4 are held by the substrate 13 of the belt 12, at a fixed, intervening interval. This interval is dimensioned to permit the electrodes l to respectively overlie electrical nodes (not shown) on the body 3 of a wearer 16 as shown in FIG. 8. The belt 12 is held in place by tension developed by connectors, e.g. hook-and-loop fastening means, once positioned on the body 3. While a narrow belt 12 is depicted in FIG. 8, a wider belt or vest 15 could carry three, four or more electrodes 1 as shown in FIG. 9.

In either of the cases of FIGS. 8 or 9 the capacitive pickup electrode performs with a better signal-to-noise ratio with an enhancer 28 present between the electrode l and the body 3 or surface from which a signal is being obtained.

An advantage of the invention is that multiple pick-up electrodes can be assembled in a preformated, fixed array that can be fitted to the body collectively, as a unitary assembly, much as in the manner of donning an article of clothing. This permits a wearer to be “fitted-up” for electrical field measurement in a very short period of time. Data acquisition can readily be suspended and resumed by the simple act of removing and then re-donning the pre-assembled array. No components need be consumed in this process. Optionally, the wearing apparel may be provided with a hygienic disposable liners of moisture permeable material.

The electrodes 1 of such a piece of apparel as shown in FIG. 9 may feed signals to a radio transmitter 19 carried by the wearer 16. In this manner an especially convenient form of telemonitoring can be achieved.

While the use of an enhancer has been largely demonstrated with reference to a capacitive electrode, the enhancer of the invention is also effective with ohmic electrodes. It is particularly effective with ohmic electrodes when CLP is employed as the cushioning material within the enhancer.

### CONCLUSION

The foregoing has constituted a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure that has been provided herein.

1. A bio-electrode for sensing body electrical fields present at a location on the surface of such body, said bio-electrode being fitted with an enhancement layer on the body-side of the electrode wherein the enhancement layer comprises a superabsorbent material which:
a) has electrical conductivity when moistened with water to provide electrical conduction between the body and the electrode;
b) has the capacity to contain water; and
c) emanates water through its outer body-facing surface when water is contained therein to thereby quiet tribo-electric effects at the interface between the enhancement layer and a body that is a source of an electrical field.

2. A bio-electrode for sensing body electrical fields present at a location on the surface of such body, said bio-electrode comprising:
a) a capacitive electrode plate;
b) an insulative dielectric layer positioned against the electrode plate on the body-side of the electrode plate, and being fitted with an enhancement layer on the body-side of the electrode wherein the enhancement layer;
c) has electrical conductivity when moistened with water to provide electrical conduction between the body and the electrode;
d) has the capacity to contain water; and
e) emanates water through its outer body-facing surface when water is contained therein to thereby quiet tribo-electric effects at the interface between the enhancement layer and a body that is a source of an electrical field; and wherein the enhancement layer is positioned against the dielectric layer on the body-side.

3. A bio-electrode as in any of the previous claims wherein the enhancement layer has an outer body-facing engagement surface that is non-adhesive but non-slippery so as to engage with a surface whose field is to be sensed sufficiently to resist displacements at the enhancement layer to body surface interface when the electrode is subjected to slight displacement forces.

4. A bio-electrode as in any of the previous claims wherein the enhancement layer comprises a “cushioning” character that allows for slight displacements of the electrode with respect to the body without significant disruption of the mechanical interfaces between the body and the enhancement layer, and between the enhancement layer and the electrode.

5. A bio-electrode as in any of the preceding claims wherein the enhancement layer, when water is contained therein, slowly releases an effective quantity of water into the body-to-electrode interface to provide enhanced electrical conductivity at such interface.

6. A bio-electrode as in any of the preceding claims wherein the enhancement layer comprises a superabsorbent material that is selected from the group consisting of sodium polyacrylate/polyacrylamide copolymer and potassium polyacylate/polyacrylamide copolymer and mixtures thereof.

7. A bio-electrode as in claim 6 wherein the copolymer is in the form of granules and is contained within a water permeable containment layer.

8. A bio-electrode as in claim 7 wherein the containment layer is a fabric textile.

9. A bio-electrode as in any of the preceding claims wherein the enhancement layer displays a bulk resistance between the electrode and a body of less than 10 Megohms.

10. A bio-electrode as in any of the preceding claims in combination with clothing layers overlying a body wherein the enhancement layer is applied over the clothing layers.

11. A bio-electrode as in any of the preceding claims wherein the enhancement layer's electrical conductivity, when wetted with water, principally arises from ionic conductivity.

12. A bio-electrode as in claim 11 wherein the enhancement layer comprises a conductive fluid selected from the group consisting of a mild aqueous solution of a salt selected from the group consisting of sodium chloride, potassium chloride, calcium chloride, magnesium chloride, lithium chloride and mixtures thereof, said solution having enhancement layer-compatible and body-compatible properties.

13. A bio-electrode as in any of the above claims wherein the electrode is a capacitive electrode with a dielectric layer coupled to an electronic sensing circuitry means designed to form a closed-circuit voltage divider network with the body and the location on the surface of the body to be sensed and wherein such bio-electrodes and electronic sensing circuitry means operate in the region of the relationship C=eA/d where capacitance C is relatively insensitive to variations in separation gap d between the electrode and the body, A being the area of the capacitive electrode and e being a factor that is dependent on the dielectric constant of the dielectric.

14. A bio-electrode as in claim 13 wherein the rate of decrease in capacitance 'C with an increase in its separation d from a body to which it is capacitively coupled is equivalent to a 0.1 mm displacement of the electrode causing less than a 50 percent change in the value of C.

15. A bio-electrode as in claim 13 wherein the electrode is provided with an insulating, dielectric layer of a thickness that precludes the electrode from providing a capacitance of more than 20 picoFarads/square centimeter.

16. A bio-electrode as in claim 13 wherein the electrode is provided with an insulating, dielectric layer of a thickness that precludes the electrode from providing a capacitance of more than 10 picoFarads/square centimeter.

17. A bio-electrode as in claim 13 wherein said electronic sensing circuitry means comprises an input series capacitance of less than substantially 20 picoFarads placed in series with the electrode to make the signal pickup relatively non-dependent on the intimacy of the electrode contact with the body.

18. A bio-electrode as in any of claims 13, 14, 15, 16 or 17 wherein the electronic circuit sensing means has an input capacitance of less than 25 picoFarads.

19. A bio-electrode as in any of claims 13, 14, 15, 16 or 17 wherein the electronic circuit sensing means has an input capacitance of less than 10 picoFarads.

20. A bio-electrode as in any of claims 13, 14, 15, 16 or 17 wherein the electronic circuit sensing means has an input capacitance of less than 5 picoFarads.
